(19) United States
(12) Patent Application Publication
(10) Pub. No.: US 2015/0157408 A1
(43) Pub. Date: Jun. 11, 2015

(54) METHOD AND APPARATUS FOR AUTOMATED CONTROL AND MULTIDIMENSIONAL POSITIONING OF MULTIPLE LOCALIZED MEDICAL DEVICES WITH A SINGLE INTERVENTIONAL REMOTE NAVIGATION SYSTEM

(71) Applicant: STEREOTAXIS, INC., St. Louis, MO (US)

(72) Inventors: Raju R. Viswanathan, St. Louis, MO (US); Walter M. Blume, Webster Groves, MO (US)

(21) Appl. No.: 14/565,246

(22) Filed: Dec. 9, 2014

Related U.S. Application Data
(60) Provisional application No. 61/913,908, filed on Dec. 9, 2013.

(51) Int. Cl. A61B 19/00; A61B 8/12; A61B 5/06

(52) U.S. Cl. CPC A61B 19/22; A61B 5/06; A61B 8/12

(57) ABSTRACT

Methods are provided for automatically actuating and positioning a localized second medical device in multiple spatial dimensions in a subject anatomy with a remote medical navigation system in coordination with a localized first medical device that is also actuated by the remote navigation system. After an initial calibration step, an exemplary method comprises:

(a) navigating the first medical device with the remote navigation system,
(b) constructing a cost function based on the spatial coordinates of the first and second medical devices, minimizing it and computing and automatically applying updates to the configurational degrees of freedom of the second medical device.

### CROSS-REFERENCE TO RELATED APPLICATIONS

This application claims the benefit of U.S. Provisional Application No. 61/913,908, filed on Dec. 9, 2013. The entire disclosure of the above application is incorporated herein by reference.

### FIELD

This invention relates to remotely controlled steering and navigation, as well as automated tracking of medical devices through a subject anatomy with a remote navigation system for interventional medicine, where a first or primary medical device is remotely steered by the remote navigation system using spatial location feedback from the medical device, and at least a second, accessory interventional medical device capable of imaging is automatically steered by the same remote navigation system, with the remote navigation system also receiving spatial location feedback from the second medical device, the devices being controlled such that the steering of the two devices by the remote navigation system occurs in a generally coordinated manner.

### BACKGROUND

Remote navigation systems for minimally invasive interventional procedures have been recently developed with different forms of actuation and have become commercially available to assist with a variety of minimally invasive interventional medical procedures. As an example, the Niobe© magnetic navigation system manufactured by Stereotaxis, Inc. of St. Louis, Mo., USA, consists of a set of magnets in a procedure room together with control hardware and software and a user interface; the magnetic navigation system is integrated with an X-ray system to receive X-ray image data from the latter. This remote navigation system can be operated and controlled from a control room outside the procedure room, thereby limiting X-ray radiation exposure to physicians operating the system and permitting the performance of an interventional medical procedure in most part from outside the procedure room. Patients generally benefit from the convenience of an overall faster procedure and correspondingly reduced X-ray exposure.

A remote navigation system may be interfaced with a localization system that determines the spatial location of a medical device within a subject anatomy, permitting the possibility of automatically steering and navigating the medical device to a desired set of target locations using closed loop feedback control. An example of an interventional medical device localization system is the CARTO® Electrophysiology localization system manufactured by Biosense-Webster, Inc. of Diamond Bar, Calif. that localizes various catheter and sheath devices incorporating location sensors and/or electrodes for applications in Electrophysiology. Another example of a localization system is the EnSite NavX™ system manufactured by St. Jude Medical of St. Paul, Minn.

Furthermore, it can be useful in many procedures to use an accessory interventional imaging device, such as for example an Intra-Cardiac Echography (ICE) catheter that captures ultrasound images by means of an ultrasound transducer mounted on the distal section of the device. In one embodiment, such a device can have its transducer mounted along the side of the device, creating a fan-like viewing window that emanates from the side of the distal tip section of the imaging device. The accessory interventional imaging device can be endowed with a location sensor interfaced to a localization system for spatially localizing the accessory device in terms of spatial position and orientation. The image data from this imaging device can also be integrated with the localization data so that the obtained image is automatically registered to the localization system coordinates.

Cardiological interventional procedures can benefit greatly from the use of such an integrated system. In the clinical application of interventional cardiac electrophysiology, a catheter-based medical procedure is performed to record electrical activity over the interior (endocardial) heart surface, permitting visualization of electrical activity and any abnormalities or arrhythmias if they exist. Subsequently, a catheter-based ablation procedure (where the ablation catheter is a primary medical device) is performed to ablate and isolate regions of the endocardial surface that function as sources of abnormal electrical activity. This type of procedure can take a long time when performed manually and requires a significant level of skill on the part of the interventional physician. In this context, a remotely operated medical navigation system can offer significant benefits especially if at least a part of the procedure can be efficiently automated, leading to reduced procedure time, reduced X-ray exposure for both physician and patient, and reduced discomfort to physician and patient. Furthermore, the ultrasound image data from the accessory imaging device can provide additional visual feedback to the physician regarding the anatomical positioning of the primary medical device and the ablation process.

Additionally, in some instances it may be useful to “park” or station the secondary medical device or imaging catheter at one location and orientation, and use the visual feedback provided by the imaging catheter to position the primary medical device or to adjust its position. In this type of application, it is advantageous to be able to make fine, controlled movements of the primary medical device, for which a remote navigation system is ideal. Thus, either device can be controlled and suitably steered in coordinated fashion from the remote navigation system where all the geometric information needed for such steering operations is maintained and utilized in the control scheme. While presently available remote navigation systems for interventional medicine can steer or control single medical devices, there are none at present that can steer multiple devices in coordinated fashion in the presence of a variety of multidimensional geometric constraints. Such constraints which are useful in practice could include, without limitation, one or more of the following: maintaining distance between appropriate points on separate medical devices, maintaining the position of a first medical device within an arbitrarily oriented viewing plane of a second medical device distance by control of either medical device, maintaining the orientation of a first medical device with respect to the viewing plane of a second medical device, maintaining the distance between a first medical device and a line in the viewing plane of a second medical device, and so on. There are at present no methods that implement such generalized control of multiple medical devices from a single remote navigation system. The disclosure of the present invention provides methods for achieving such objectives.

### SUMMARY

Embodiments of the present invention relate to methods of remotely controlling steering and navigation, as well as methods for the automated tracking of medical devices through a subject anatomy with an interventional remote navigation system. According to the methods taught in the preferred embodiment of the present invention, a first or primary medical device is remotely steered by the remote navigation system using spatial location and orientation feedback from the medical device, and at least a second, accessory interventional medical device capable of imaging is automatically steered by the same remote navigation system, in such a manner that the steering of the second device occurs in coordinated manner with the first and satisfies a number of multidimensional geometric constraints. The remote navigation system also receives spatial location feedback from the second medical device.

Multiple geometric constraints and conditions can be defined in terms of the positions and orientations of the two devices and incorporated into a cost function. The cost function is designed to be minimized when the geometric constraints are simultaneously satisfied to the best extent possible. In general, perfectly satisfying multiple such constraints simultaneously is not always feasible since some of the constraints may be in conflict such that one constraint may not be perfectly satisfied while another one is; thus the optimization process is designed to find a solution that permits simultaneous satisfaction in an optimal manner. When it is desired to implement the requirement of strong satisfaction for some constraints that are deemed more important than others, appropriately higher weights can be assigned to those constraints in the construction of the cost function. Geometric constraints on two devices that are useful in practice could include, without limitation, one or more of the following: maintaining distance between appropriately selected locations or points on separate medical devices; maintaining the position of a first medical device within an arbitrarily oriented viewing plane of a second medical device distance by control of either medical device; maintaining the orientation of a first medical device with respect to the viewing plane of a second medical device; maintaining the distance between a first medical device and a line in the viewing plane of a second medical device, and so forth. It is important to note that depending on the application, there could be a range of geometric constraints that could be useful to implement, and the techniques of the embodiments of the present invention apply without limitations in such cases.

Additionally, a computational model of the response of the response of at least one of the devices is used in the formulation of the cost function and its optimization process. The case where the ICE catheter is controlled is explicitly described in terms of a computational model for the device that is used in the optimization process. Without loss of generality, this process can be extended to a multiplicity of devices without departing from the scope of the present invention.

Furthermore, since the geometric constraints are generally based on positions and orientations of multiple devices, either device could be controlled or steered based on the methods of the embodiments of the present invention. For exemplary purposes, the disclosure herein treats the case of a (first) mapping/ablation catheter that it is desired to track with a (second device) imaging catheter. Both devices are driven from a single remote navigation system, while the steering modality generally could be different for the two devices; thus the first device could be steered magnetically, while the second device could be actuated mechanically. A range of other actuation schemes could be used, and likewise a larger multiplicity of devices could be deployed in the clinical application and controlled according to the teachings of the present disclosure. It is to be noted that the complementary situation where the second device is stationed or parked in a particular position with a given viewing plane orientation, and it is desired to move the first device into the viewing plane of the second device, can also be approached in a manner evident from the disclosure of the embodiments of the present invention. The geometric constraints that are desired to be satisfied are encoded in suitably defined terms of a cost function where each such constraint or term generally depends on position/orientation variables of multiple devices. In this manner, the same cost function can be used to relatively position any of the devices.

Thus, generally the embodiments of this invention apply to relative positioning of multiple medical devices that are driven from or actuated by a single remote navigation system, under generally multidimensional geometric constraints. For instance, the coordinated movement of two devices such that either one can follow the other under control from the same remote navigation system is an aspect taught in the present disclosure. The same high-level control computer of the remote navigation system can generally contain navigation or steering control algorithms to drive both devices in coordinated fashion. These and other advantages are further described and expanded on in the disclosure below.

### BRIEF DESCRIPTION OF THE DRAWINGS

FIG. 1 is a schematic illustration of an Intra-Cardiac Echography (ICE) catheter in a right atrial heart chamber, showing the imaging plane of the device, and a catheter device in the left atrial heart chamber that lies in the imaging plane of the ICE catheter.

FIG. 2 is an illustration of the degrees of freedom associated with the ICE catheter that are used in the formulation of the computational model.

FIG. 3 shows a typical geometrical disposition of an ICE catheter and a magnetic ablation catheter and labels associated variables that are used in the automated control scheme of embodiments of the present invention.

FIG. 4 is a schematic depiction of a system diagram of a preferred embodiment in accordance with the present invention illustrating various system components and communication paths.

FIG. 5 shows a user interface of a preferred embodiment of the present invention where a localized magnetic ablation catheter tip is graphically rendered in a rendering of a three dimensional scene along with a portion of a surface schematically representing a portion of cardiac anatomy. The viewing/imaging plane of the ICE catheter is also shown in the three dimensional scene. A portion of the screen also shows the image obtained from the ICE catheter. The ablation catheter in this Figure is outside the viewing plane of the ICE catheter. The user employs a calibration procedure that subsequently moves the ICE catheter so that the ablation catheter is contained in the viewing plane.

FIG. 6 shows a user interface of the preferred embodiment of the present invention at the end of a calibration procedure that moves the ICE catheter such that the tip portion of the ablation catheter is contained in the viewing plane of the ICE catheter. The tip of the localized magnetic ablation catheter is graphically rendered in a rendering of a three dimensional scene along with a portion of a surface schematically representing a portion of cardiac anatomy and the viewing/imaging plane of the ICE catheter is also shown in the three dimensional scene; the ablation catheter is contained in the viewing plane of the ICE catheter after the user has completed a calibration procedure whereby the user moves the ICE catheter under visual feedback in the three dimensional scene until the ablation catheter is seen to lie in the viewing plane of the ICE catheter. This move is performed by manually controlling a suitable navigation drive unit. A portion of the screen also shows the image obtained from the ICE catheter where the ablation catheter tip is visible in the image plane.

FIG. 7 shows a user interface of the preferred embodiment of the present invention where the display of the three dimensional scene shows an ablation catheter tip that has moved away from the viewing plane of the ICE catheter. The user can initiate automated tracking by clicking on a “Start” button on the graphical user interface. Upon initiation, the methods of the preferred embodiments of the present invention are used to automatically steer and move the various degrees of freedom of the ICE catheter until the ablation catheter tip lies within the viewing plane of the ICE catheter and a variety of geometrical constraints have been satisfied in optimal manner.

FIG. 8 illustrates a user interface of the preferred embodiment of the present invention where the display of the three dimensional scene shows an ablation catheter tip that is visible in the viewing plane of the ICE catheter after the ICE catheter has been suitably moved into an appropriate viewing position by automatic control of its degrees of freedom according to the teachings of the present disclosure.

### DETAILED DESCRIPTION

This disclosure describes a set of methods for automatically navigating at least one medical device in a coordinated manner with another medical device such that the two medical devices end up simultaneously satisfying a generally multidimensional set of geometric constraints in an optimal manner. In particular and for purely exemplary purposes, consider the case of a first medical device in the form of an ablation catheter that is steered with a remotely operated magnetic navigation system, and a second medical device in the form of an Intra-Cardiac Echography (ICE) ultrasound catheter that is used to observe or visualize among others the distal tip of the ablation catheter. The ICE catheter has an ultrasound transducer or set of transducers mounted such that the ultrasound image field of view is a fan or wedge shaped region emanating from the side of the distal portion of the ICE catheter. It is desired to maintain the first medical device within this fan-shaped field of view of the ultrasound/ICE catheter.

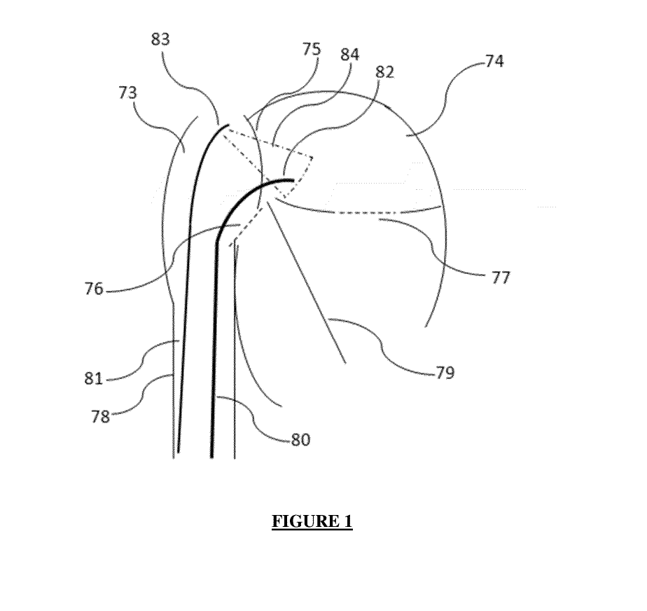

An exemplary application and geometrical disposition is shown in FIG. 1. Referring to FIG. 1, an ablation catheter 80 and an ultrasound ICE catheter 81 are shown inserted in the Inferior Vena Cava (IVC) 78 of a subject anatomy, where the cardiac anatomy is schematically represented showing the Right Atrial chamber 73, the Left Atrial chamber 74, the atrial septum 75, the ventricular septum 79, and Tricuspid and Mitral valves 76 and 77 respectively (the valves indicated as respective dotted lines). As shown, the distal tip portion 82 of the ablation catheter 80 is inserted into the left atrium via a trans-septal puncture. The catheter 80 is used to perform an ElectroPhysiology (EP) procedure where intracardiac electrograms are recorded with the catheter to diagnose arrhythmic regions of the heart and an RF ablation process is used to electrically isolate undesirable electrical nodes in the cardiac anatomy. The distal portion 83 of the ICE catheter 81 has ultrasound transducers mounted laterally to generate a fan-like ultrasound beam 84 of small thickness, within which it is desirable to view the distal tip portion 82 of the ablation catheter 80. The fan-like beam 84 is the viewing plane associated with the ICE catheter.

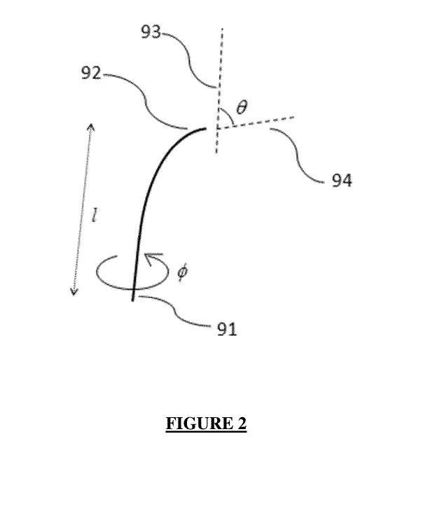

FIG. 2 illustrates the degrees of freedom associated with the ultrasound ICE catheter. The base 91 of the catheter can be moved in and out or advanced and retracted at a given location. The length of catheter beyond this location is denoted by the variable 1, and is the length degree of freedom. Further the catheter can rotate about its long axis by a rotation angle φ (from some reference configuration) as shown by the circular motion indicated by the circular arrow at the base of (a portion of) the catheter in FIG. 2. The distal tip region 92 can deflect by an angle θ with respect to the base, as shown by the angle between dotted lines 93 and 94 that are parallel to the base orientation and the distal tip orientation, respectively.

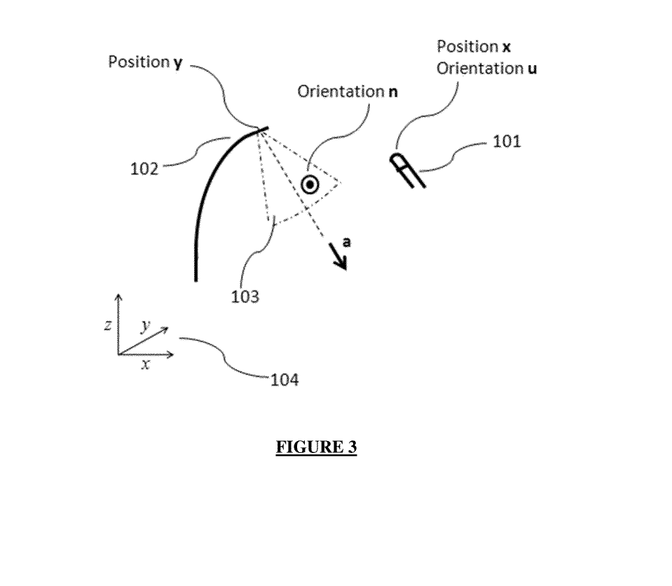

The detailed geometry of the two devices in the exemplary embodiment of the present invention is shown in FIG. 3. In this Figure, the tip of the ablation catheter is shown as 101 and has position and orientation vectors labeled by x and u respectively (so u is a unit vector denoting tip tangent). The ultrasound beam fan 103 emanates from the side of the ICE catheter 102 in the distal tip region of the ICE catheter and is centered at position y on the distal tip portion of the ICE catheter. The fan centerline is described by unit vector a, as shown in the figure. The spatial orientation of the ultrasound fan is described by the normal n to the plane of the fan, as shown in the figure pointing outward from the viewing plane and the plane of the page. A coordinate system 104 is shown with local x, y and z axes attached to the base of the catheter.

In practice, the ICE catheter length that is modeled starts at a known anatomical location, for instance where the Inferior Vena Cava enters the right atrium. For convenience, we consider the case where the entire length of catheter further to this anatomical location is deflectable, except for a rigid tip portion. If the maximum deflectable length of the ICE catheter is L, the (deflectable) length of catheter l inserted in the right atrium and that we will model usually satisfies l<L. A calibration procedure is followed for the ICE catheter before tracking another device is initiated. The ICE catheter is driven by passing it though a drive mechanism (such as the Vdrive™ unit manufactured by Stereotaxis, Inc. of St. Louis, Mo.) where rotations of appropriate catheter handle regions are generated by suitable gripping/clamp mechanisms driven by drive motors. These rotations can result in rotations of the catheter shaft and deflections of the ICE catheter distal portion generated by pull-wires inside the catheter. Likewise, catheter insertion/retraction is generated mechanically by translation of the gripping mechanisms over a length range, again driven by a suitable drive motor and appropriate motion transmission mechanisms. Further details of such drive mechanisms for remote device manipulation are disclosed in US patent application 2009/00105645, “Apparatus for selectively rotating and/or advancing an elongate device”, and US patent application 2010/0298845, “Remote manipulator device”, attached here for reference.

Since the ICE catheter is a localized device (for instance by means of a suitable sensor mounted in its tip region), the position and orientation of the tip as well as the catheter's imaging/viewing plane (defined in terms of its normal n) are known quantities. The ICE catheter's base orientation is also assumed known (for instance, it may be assumed to be along the z axis in the case where it enters the right atrial chamber along the IVC). Further, the calibration process allows the user to identify the catheter tip at or near the point of entry into the chamber, which defines a reference position for the length of the catheter. Since the catheter tip is localized, an initial reference position for the rotation angle φ (φ=0) is also defined and subsequent φ values can be determined from tip orientation and the known base direction. Likewise, from the known tip orientation the reference deflection angle θ is also known. The current (initial) motor positions (determined from appropriate encoders) for the device manipulation drive mechanism for the respective degrees of freedom can also be recorded as reference values.

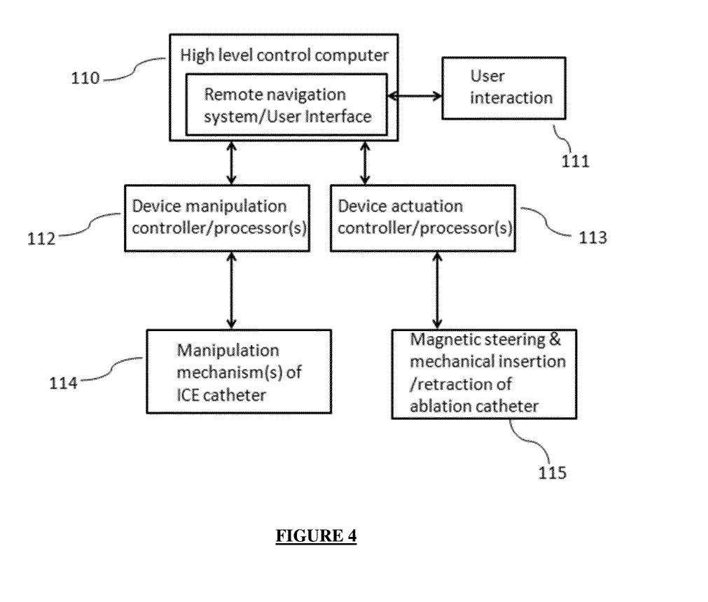

FIG. 4 is a high-level system diagram describing one version of a system architecture for implementing the teachings of the present disclosure. The remote navigation system has a high level control computer 110 that runs, among others, the remote navigation user interface which offers user interaction 111 permitting a user to view various displays and operate an assortment of controls. Some of these control inputs generally conceivable as user interaction means 111 could be, additionally to graphical user interface tools such as buttons, sliders, clicking on various visual displays, etc., in the form of hardware such as a mouse, joystick, or other input devices with a combination of joystick and buttons. Such input devices and user interaction means are known widely in the prior art and are not described in detail here. The high level control computer 110 of the remote navigation system interfaces with a device manipulation controller or controllers 112, and with a set of device actuation controllers/processors 113, respectively to manipulate or generally actuate an ICE imaging catheter, and a magnetic ablation catheter. The device manipulation controller(s) 112 drive (servo) motors in a manipulation mechanism 114 which controls and drives the handle of the ICE catheter, and which can generally rotate, deflect, or advance/retract the catheter. Encoders in the motors keep the manipulation controller(s) appraised of motor positions and/or velocities etc. Likewise the device actuation controllers/processors 113 drive motors associated with moving magnets to create an appropriately directed magnetic field to steer the ablation catheter device, as well as a motor to advance or retract the ablation catheter, all represented as steering and insertion/retraction operations 115. Both devices in this case (ICE catheter and ablation catheter) are localized and their localization information is available to the high level control computer 110 of the remote navigation system via interfacing to a localization system (not shown). Thus the single high level control computer 110 has access to all the relevant information needed to position either device so that appropriate multidimensional constraints are satisfied, using methods to be described below. It is to be noted that while the exemplary description here is for the case of two devices, a larger multiplicity of devices can also be controlled and steered or positioned according to the teachings of the present disclosure.

The ICE catheter generally has a rigid tip of known length t (for instance, t can be in the approximate range of 1-2 cm). Further, we make the assumption that the deflectable portion of the ICE catheter has uniform physical properties. In this case, the deflection of the catheter that is generated by tension in a pull-wire follows a circular arc. If a deflectable length of catheter l extends from the base, and the tip is deflected by an angle θ relative to the base, the tip location y can then be written in local coordinates (in the coordinate frame shown in FIG. 3, with the base of the catheter as origin) explicitly in terms of components as

$${y}_{l} = ( \frac{l}{θ} ( 1 - cos θ ) cos φ , \frac{l}{θ} ( 1 - cos θ ) sin φ , \frac{l}{θ} sin θ ) + t ( sin θ cos φ , sin θ sin , cos θ ) \qquad ( 1 )$$

The fan centerline vector a can likewise be written, in the same set of local coordinates, as

aₗ=(cos θ cos φ,cos θ sin φ,−sin θ) (2)

while the normal vector n to the imaging plane can be written in local coordinates as

nₗ=(sin φ,−cos φ,0). (3)

The local x, y and z axes may be expressed in terms of global coordinates by a suitable rotation, and likewise global position coordinates may be written in terms of local coordinates by an appropriate rotation and translation. Generally, we can write

y=Myₗ+p (4)

where M is a rotation matrix and p is a translation vector (for instance, the point of entry for the catheter base) that together implement the coordinate transformation from local to global coordinates; likewise global expressions for a and n can be written respectively as a=Maₗ and n=Mnₗ.

The approach we take to implementing the simultaneous satisfaction of constraints is cost function optimization. Considering the geometry of the two devices explicitly shown in FIG. 3, it is generally desirable in many applications to optimally view the ablation catheter within the field of view of the ICE catheter. One suitable constraint is thus that the tip x of the ablation catheter lies within the plane of the imaging catheter. This constraint may be encoded in the form of a dimensionless cost term

$${C}_{1} = \frac{{( n \cdot ( x - y ) )}^{2}}{{\vert x - y \vert}^{2}} \qquad ( 5 )$$

where the numerator goes to zero when the vector (x−y) lies in the imaging plane and is therefore perpendicular to the plane normal n.

We also require that the ablation catheter lie close to the centerline of the ultrasound fan beam. Thus, we would like to minimize the distance d from the line represented by unit vector a and the ablation catheter tip, x. It can be shown that this distance can be written in squared form as

d²=|x−y|²−(a·(x−y))² (6)

and accordingly we define the normalized (dimensionless) cost term

$${C}_{2} = \frac{{\vert x - y \vert}^{2} - {( a \cdot ( x - y ) )}^{2}}{{\vert x - y \vert}^{2}} \qquad ( 7 )$$

The cost term C₂ goes to zero when the distance from the ablation catheter tip to the fan centerline defined by a goes to zero. Next, we would like to impose the constraint that the distal tip of the ablation catheter, with tip orientation described by unit vector u, is maximally contained within the viewing plane. The dimensionless cost term

C₃=(n·u)² (8)

goes to zero when u is perpendicular to n, and thus lies in the imaging plane.

We then define a total cost function as a weighted sum of the three cost terms defined above:

$$\begin{matrix}{C}_{tot} = {w}_{1} {C}_{1} + {w}_{2} {C}_{2} + {w}_{3} {C}_{3} \\ = {w}_{1} \frac{{( n \cdot ( x - y ) )}^{2}}{{\vert x - y \vert}^{2}} + {w}_{2} \frac{{\vert x - y \vert}^{2} - {( a \cdot ( x - y ) )}^{2}}{{\vert x - y \vert}^{2}} + {{w}_{3} ( n \cdot u )}^{2}\end{matrix} \qquad ( 9 )$$

where the weighting coefficients w₁, w₂ and w₃ are pre-defined.

With a cost function defined, the process of finding an optimal solution can proceed as follows. We will treat the ablation catheter position as given and vary the ICE catheter degrees of freedom to find a tracking solution where the ICE catheter viewing plane moves until it settles into a configuration where the ablation catheter lies in the viewing plane and minimizes the total cost function in equation (9). The ablation catheter is also steered and navigated to various target locations by the remote navigation system. As a localized device, its tip position x is available to the remote navigation system by appropriate interfacing to a localization system. Given an ablation catheter position that it is desired to track, a check is performed to see whether the ultrasound fan is rotated more than 90 degrees away from the ablation catheter tip position. If it is, the ICE catheter is rotated in an appropriate direction (clockwise or counter-clockwise) so that the ablation catheter position is on the same side as the ultrasound fan beam with respect to the ICE catheter. Next, a gradient descent procedure is used to adjust the ICE catheter degrees of freedom. Given a current configuration of the catheter (and thus its current degrees of freedom θ, l and φ), an update to the degrees of freedom is computed and applied to the ICE catheter by the remote navigation system via the catheter manipulator mechanism and its controller.

From expression (9) for the total cost function, partial derivatives can be obtained either analytically using equations (1), (2), (3) and (4), or computationally by making small changes computationally in the respective degrees of freedom and evaluating expressions:

$$\frac{\partial {C}_{tot}}{\partial θ} ≅ \frac{{C}_{tot} ( θ + Δθ ) - {C}_{tot} ( θ )}{Δθ} , \\ \frac{\partial {C}_{tot}}{\partial φ} ≅ \frac{{C}_{tot} ( φ + Δφ ) - {C}_{tot} ( φ )}{Δφ} , \\ \frac{\partial {C}_{tot}}{\partial l} ≅ \frac{{C}_{tot} ( l + Δ l ) - {C}_{tot} ( l )}{Δ l} + \frac{θ}{l} \frac{{C}_{tot} ( θ + Δθ ) - {C}_{tot} ( θ )}{Δθ} \qquad ( 10 )$$

where the last term in the equation above arises from the fact that when a length change is made at constant curvature of the deflected catheter, it also changes the tip orientation at the same time. Since equations (1)-(4) explicitly determine the various variables in terms of the degrees of freedom of the ICE catheter, the derivatives in equations (10) can be determined numerically from their approximate representations on the right hand side, given the current catheter configuration.

In one embodiment the current configuration's degrees of freedom (θ, l and φ) can be determined from knowledge of the (localized) current tip position y and the known base entry position p, and fitting the degrees of freedom from equations (1) and (4) to determine them. In an alternate embodiment, an internal map is maintained between appropriate drive mechanism motor positions and the corresponding degrees of freedom (for example and for illustration purposes only, a 90-degree turn of an appropriate motor shaft may correspond to a 25-degree deflection in the ICE catheter tip, etc.), and this map is used together with knowledge of current motor positions or related drive mechanism variables to estimate a current set of values for the ICE catheter degrees of freedom. In yet another alternate embodiment, a combination of these two methods could be used to find a best-fit estimate of the catheter degrees of freedom. Once known, the current values of the degrees of freedom can be used to compute cost function derivatives according to equations (10) above.

With the derivatives from equation (10) in hand, updates to the degrees of freedom are computed from the following equations:

$$δθ = - {k}_{θ} \frac{\partial {C}_{tot}}{\partial θ} \\ δφ = - {k}_{φ} \frac{\partial {C}_{tot}}{\partial φ} \\ δ l = - {k}_{l} \frac{\partial {C}_{tot}}{\partial l} \qquad ( 11 )$$

where it has been determined by the inventors that in a preferred embodiment, values for the coefficients k_{θ}, k_{φ} and kₗ respectively in the ranges 0.2<k_{θ}<0.8, 0.1<k_{φ}<0.7, and 200<kₗ<1200 yield good convergence in a fast and efficient manner, with angles measured in degrees and length in millimeter (mm) units. Alternate embodiments could use other units and/or other ranges of values for these adjustment coefficients. The changes in the ICE catheter degrees of freedom, equations (11) above, are implemented by converting them to appropriate motor movements that drive the respective degrees of freedom. This conversion can be computed and applied by the high-level control computer of the remote navigation system or in a preferred embodiment by the device manipulation controller(s).

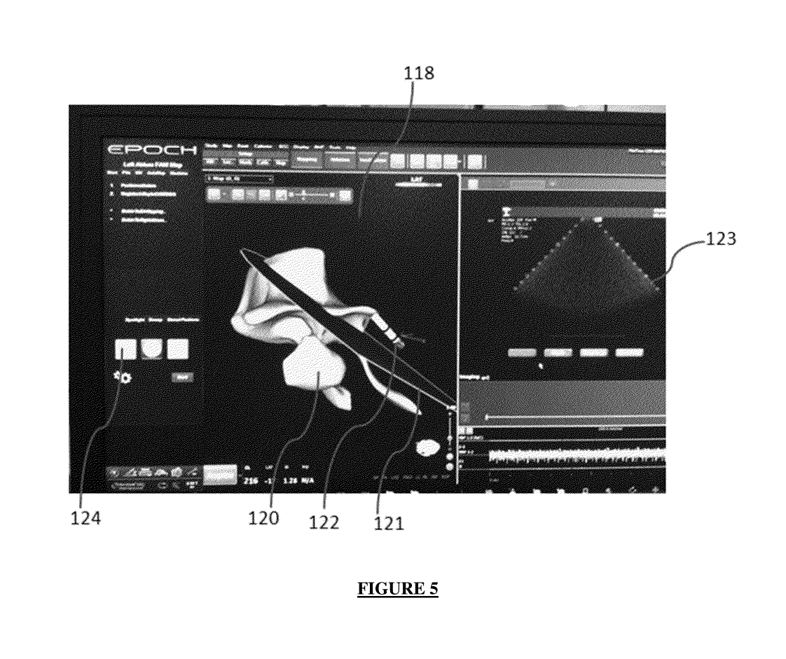

FIG. 5 is an illustration showing a user interface of the preferred embodiment of the present invention including a three dimensional graphical display window 118 in the left half of the screen where a representation of an anatomical surface 120 is displayed, along with the localized ablation catheter tip 122 and the ICE catheter imaging plane 121. The display 123 of the ICE imaging catheter is shown on the portion of the screen on the right where the fan-like region imaged by the ultrasound beam of the ICE catheter is visible. As an initial step, a user invokes a calibration routine by clicking on a graphical user interface button 124 with a click of a computer mouse. Upon doing so, as a next step the user moves the ICE catheter imaging plane by manipulating the ICE catheter drive mechanism, for example via joystick control (not shown), to cause the ICE catheter to rotate, deflect or advance/retract suitably, with the intent of bringing the ablation catheter tip into the imaging plane of the ICE catheter. Because the ICE catheter is also a localized device, its movements and in particular the location and orientation of the imaging plane are known, and so the three dimensional graphic display is suitably updated in real time as the user manipulates the ICE catheter via the joystick.

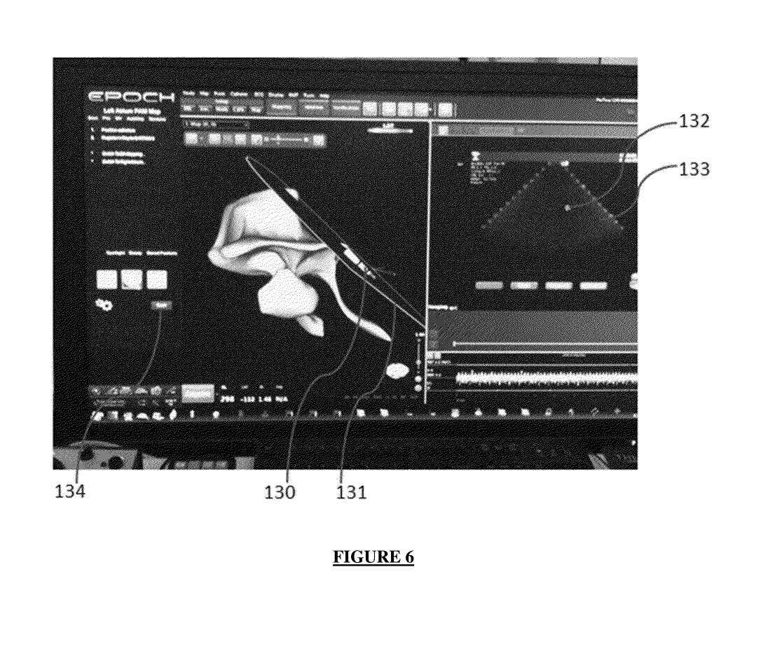

As visible in FIG. 6, the ablation catheter tip 130 lies within the imaging plane of the ICE catheter 131 as seen in the three dimensional graphic window in FIG. 6. The ICE catheter image 133 also shows the ablation catheter tip 132 now, providing further confirmation that the ablation catheter tip lies in the imaging plane of the ICE catheter. The user then engages a “Start” button 134 on the graphical user interface to indicate that the current ICE catheter configuration may be used as a reference or initial configuration from which further movements of the ablation catheter are to be tracked. In other words, automatic tracking of the ablation catheter by the ICE imaging catheter is now engaged, and all further movements of the ablation catheter would result in corresponding movements of the ICE catheter that are repeatedly made in iterative manner using equations (11) above. In one preferred embodiment, the iterative changes in the degrees of freedom are continuously operational as long as the automated tracking mode is engaged. In an alternate preferred embodiment, the iterative applications of changes in the degrees of freedom are stopped once the respective changes become small enough quantities that lie below pre-defined threshold values, and automatically resume once they start growing again due to further movements of the ablation catheter. In this manner, the remote navigation system becomes easy to use for the user/physician, and tracking can continue as desired automatically and with minimal user interaction.

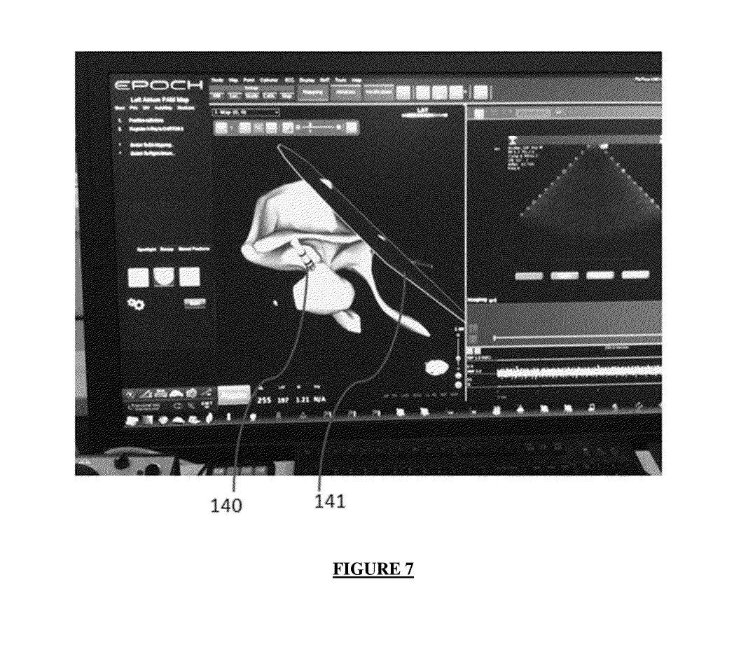

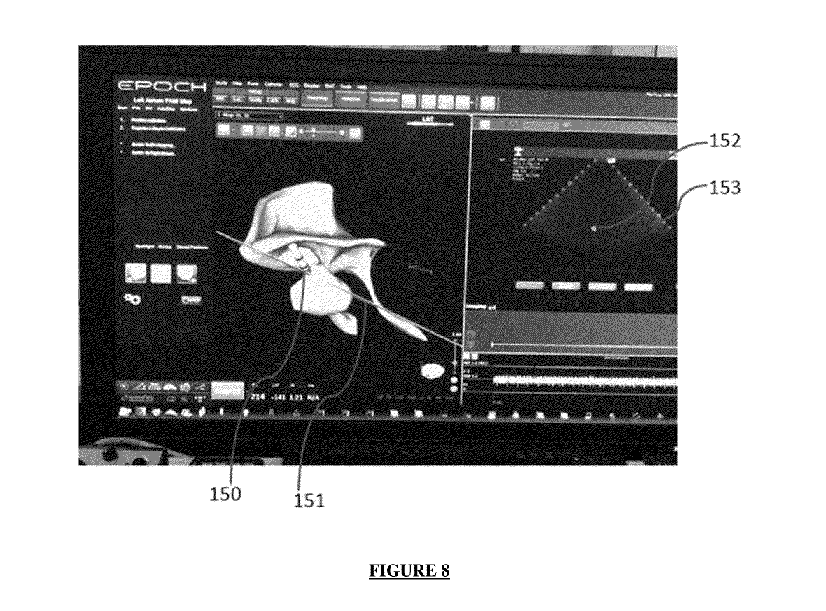

FIG. 7 shows an example where the ablation catheter tip 140 has moved away from the imaging plane 141 to a different location. FIG. 8 illustrates that with automatic tracking engaged, the degrees of freedom of the ICE catheter are automatically updated by the system until, within a few iterations, the imaging plane of the ICE catheter 151 has moved so as to contain the ablation catheter tip 150. As can be seen in the image plane 153 generated by the imaging catheter, the ablation catheter distal tip 152 lies in the imaging plane and is automatically positioned close to the centerline, as implemented by the cost function approach defined through equations (9), (10) and (11).

It has been determined by the inventors that in a preferred embodiment, the weighting factors or coefficients w₁, w₂ and w₃ can be given values in the respective ranges 0.2<w₁<2, 0.1<w₂<1, and 0.01<w3<0.3. In alternate embodiments and/or with other choices of units for the angular variables and length variable, alternate ranges of values for the coefficients could be more appropriate. In an alternate preferred embodiment, a somewhat different cost term could be used for maintaining the ablation catheter tip close to the ultrasound fan centerline. In this variation, rather than the cost term C₂ defined in equation (7) as a (normalized) squared distance, we define instead the cost term

$${C}_{2}^{′} = {( 1 - \frac{a \cdot ( x - y )}{\vert x - y \vert} )}^{2} \qquad ( 12 )$$

where the second term inside the parenthesis in equation (12) is interpreted as the cosine of the angle between the vectors a and (x−y). Thus, the cost function term (12) is zero when these vectors are perfectly aligned and the ablation catheter device lies on the centerline of the ultrasound fan beam. With this variant cost term, the rest of the cost function construction and optimization proceeds as described earlier. The total cost function is defined in a manner similar to that in equation (9) with similar weighting coefficients. For this variant or alternate form of the cost function, the inventors have determined that gradient descent coefficients k_{θ}, k_{φ} and kₗ in the respective ranges 0.5<k_{θ}<5, 0.1<k_{φ}<0.7, and 500<kₗ<2500 yield good convergence in a fast and efficient manner, with angles measured in degrees and length in millimeter (mm) units.

The advantages of the embodiments described here in detail and improvements thereupon should be readily apparent to one skilled in the art, for purposes of providing a fast and effective set of automated control algorithms for the multidimensional positioning of multiple medical devices by a single remote navigation system. The various automated positioning schemes described in the present disclosure permit actuation and steering of multiple medical devices generally in automatic fashion using device location information. Furthermore, while the specific description in this disclosure has discussed in detail the case where the imaging catheter is steered so as to track the ablation catheter, it should be obvious to one skilled in the art that the complementary situation where the imaging catheter position and viewing plane are given and the ablation catheter is steered so as to position it within the viewing plane can be automated in a manner similar to that described here, and using the same cost function approach, in an alternate embodiment. It is worth noting that the cost function terms each depend generally on both the ablation catheter tip position/orientation and the imaging catheter tip position/orientation. By having a computational model for the deflection and general configuration of either device, as for example given by equations (1)-(4) for the imaging catheter, either device can be automatically steered so as to position it in an appropriate configuration in a generalized model where the choice of which device to automatically position can be user-defined in another embodiment, while in still another embodiment the choice of which device to automatically position can be generated by the remote navigation system itself based on a set of pre-defined criteria, for instance distances from appropriate anatomical locations. The general approach disclosed in the present disclosure is that geometric constraints that are desired to be satisfied are encoded in suitably defined terms of a cost function, where each such constraint or term generally depends on position/orientation variables, also referred to as generalized spatial coordinates, of multiple devices. In this manner, the same cost function can be used to relatively position any of the devices.

While one of the devices in the description given in the present disclosure is a magnetic ablation catheter driven that is generally steered by a magnetic navigation system, the methods of the embodiments of the present invention can be extended to other actuation schemes. Thus, the method of actuation used by the remote navigation system can be any of a variety of actuation methodologies known in the art, including without limitation magnetic navigation methods, mechanical actuation, electrostrictive actuation, hydraulic actuation, etc. The remote navigation system may also use a combination of actuation modalities so that in some embodiments, multiple actuation schemes may be used by the remote navigation system. In one embodiment, the same device can be actuated with different actuation modalities; for example, a magnetic catheter can be mechanically advanced and magnetically deflected or steered. For convenience, the term “remote navigation system” in the description herein refers without limitation to any system that uses such remote actuation techniques or modalities singly or in combination, and the automated positioning algorithms of the embodiments of the present invention can drive or navigate devices that employ any or a multiplicity of such actuation methods.

Generally, the methods of the present disclosure apply to the coordinated movement and/or positioning of a multiplicity of remotely navigated or steered medical devices. A single high-level control computer that is part of the remote navigation system runs navigation algorithms designed to automate this coordinated movement and/or positioning of multiple devices. A combination of different actuation schemes may be used at the same time to control or actuate different medical devices, or even different types of movements of a single device, as may be convenient for a particular application or set of applications. Additional design considerations and/or variations that are conceived by one skilled in the art may be incorporated without departing from the spirit and scope of the disclosure. Accordingly, it is intended that the invention is not limited by the particular embodiments or forms described above, but rather by the scope of the appended claims.

What is claimed is:

1. A method for automatically actuating and positioning a localized second medical device in multiple spatial dimensions in a subject anatomy with a remote medical navigation system in coordinated fashion with a localized first medical device that is also actuated by the remote navigation system, the method comprising the steps of:
(a) calibrating an initial configuration of the second medical device,
(b) moving the first medical device by suitable actuation with the remote navigation system,
(c) computationally constructing a cost function based on the generalized spatial coordinates of the first and second medical devices,
(d) computationally minimizing the cost function by computing updates to the configurational degrees of freedom of the second medical device, and
(e) applying the computed updates to the degrees of freedom of the second medical device with the remote navigation system in order to modify its configuration.

2. The method of claim 1, where the method of actuation of the first medical device comprises the change of a magnetic field variable.

3. The method of claim 1, where the method of actuation of the second medical device comprises the change of a mechanically driven variable.

4. The method of claim 1, where the cost function construction comprises a weighted summation of multiple terms, with each term representing a distinct geometrical constraint.

5. The method of claim 1, where the computed updates to the configurational degrees of freedom of the second medical device comprise computing updates to at least one of (i) deflection, (ii) rotation, or (iii) length degrees of freedom.

6. The method of claim 1, where the computed updates to the configurational degrees of freedom of the second medical device are generated using a computational model of the second medical device.

7. A method for automatically actuating and positioning a localized imaging catheter medical device in a subject anatomy in multiple spatial dimensions with a remote medical navigation system in coordinated fashion with a localized first medical device that is also actuated by the remote navigation system, the method comprising the steps of:
(a) calibrating an initial configuration of the imaging catheter by manual adjustment of controls of the remote navigation system such that the first medical device is brought within the imaging field of view of the imaging catheter,
(b) navigating the first medical device by suitable actuation with the remote navigation system,
(c) computationally constructing a cost function based on the generalized spatial coordinates of the imaging catheter and of the first medical device,
(d) computationally minimizing the cost function by computing updates to the configurational degrees of freedom of the imaging catheter,
(e) applying the computed updates to the degrees of freedom of the imaging catheter with the remote navigation system in order to modify its configuration, and
(f) repeatedly iterating steps (c) through (e) so as to bring and maintain the first medical device within the field of view of the imaging catheter.

8. The method of claim 7, where steps (b) through (f) are repeatedly applied as the first medical device is navigated to different anatomical locations.

9. The method of claim 7, where the step of calibrating an initial configuration of the imaging catheter comprises bringing the distal tip portion of the first medical device into an ultrasound fan beam of the imaging catheter.

10. The method of claim 7, where the last step of bringing and maintaining the first medical device within the field of view of the imaging catheter comprises bringing the distal tip portion of the first medical device into an ultrasound fan beam of the imaging catheter.

11. The method of claim 7, where the method of actuation of the first medical device comprises the change of a magnetic field variable.

12. The method of claim 7, where the method of actuation of the imaging catheter comprises the change of a mechanically driven variable.

13. The method of claim 7, where the cost function construction comprises a weighted summation of multiple terms, with each term representing a distinct geometrical constraint.

14. The method of claim 7, where the computed updates to the configurational degrees of freedom of the imaging catheter comprise computing updates to at least one of (i) deflection, (ii) rotation, or (iii) length degrees of freedom.

15. The method of claim 7, where the computed updates to the configurational degrees of freedom of the imaging catheter are generated using a computational model of the imaging catheter.

16. The method of claim 13, where the weighted summation of multiple terms comprises a weighted summation of normalized, dimensionless quantities.

17. A system for automatically actuating and positioning a localized imaging catheter medical device in a subject anatomy in multiple spatial dimensions with a remote medical navigation system in coordinated fashion with a localized first medical device that is also actuated by the remote navigation system, the system comprising:
(a) a remote navigation system,
(b) a means for interfacing the remote navigation system to a localization system for localization data corresponding to the first medical device and the imaging catheter,
(c) a high level control computer for the remote navigation system, connected to a user interface for user interaction with the system,
(d) a device actuation controller that interfaces with the high level control computer for driving device actuation of the first medical device,
(e) a device manipulation controller that interfaces with the high level control computer for driving device manipulation of the imaging catheter medical device,
(f) means for changing a magnetic field applied by the remote navigation system in a subject anatomy by movement of magnets driven by the device actuation controller,
(g) means for mechanically driving changes in imaging catheter device configuration by a drive mechanism controlled by the device manipulation controller, and
(h) a computational algorithm that runs on the high level control computer for cost function construction and minimization and for generating computed updates to the configurational degrees of freedom of the imaging catheter, the cost function construction being based on the generalized spatial coordinates of the imaging catheter and of the first medical device.

18. The system of claim 17, where the imaging catheter is an ultrasound Intra-Cardiac Echography imaging catheter that generates a fan-like ultrasound beam emanating from a side of the distal tip portion of the imaging catheter.

19. The system of claim 17, where the computational algorithm for cost function construction and minimization incorporates a weighted summation of distinct cost terms, each cost term representing a different geometrical constraint.

20. The system of claim 17, where the computed updates to the configurational degrees of freedom of the imaging catheter are used to bring the distal tip portion of the first medical device within the field of view of the imaging catheter.

21. A system for automatically actuating and positioning a localized second medical device in a subject anatomy in multiple spatial dimensions with a remote medical navigation system in coordinated fashion with a localized first medical device that is also actuated by the remote navigation system, the system comprising:
(a) a remote navigation system,
(b) a means for interfacing the remote navigation system to a localization system for localization data corresponding to the first medical device and the second medical device,
(c) a high level control computer for the remote navigation system, connected to a user interface for user interaction with the system,
(d) a device actuation controller that interfaces with the high level control computer for driving device actuation of the first medical device,
(e) a device manipulation controller that interfaces with the high level control computer for driving device manipulation of the imaging catheter medical device,
(f) means for changing a magnetic field applied by the remote navigation system in a subject anatomy by movement of magnets driven by the device actuation controller,
(g) means for mechanically driving changes in second medical device configuration by a drive mechanism controlled by the device manipulation controller, and
(h) a computational algorithm that runs on the high level control computer for cost function construction and minimization and for generating computed updates to the configurational degrees of freedom of the second medical device, the cost function construction being based on the generalized spatial coordinates of the second medical device and of the first medical device.
